(19) United States
(12) Patent Application Publication
(10) Pub. No.: US 2012/0053144 A1
(43) Pub. Date: Mar. 1, 2012

(54) Cobalamin Taxane Bioconjugates For Treating Eye Disease

(75) Inventors: John R. Gebhard, Cottonwood Heights, UT (US); Dinesh Patel, Salt Lake City, UT (US)

(73) Assignee: Osiris Therapeutics, Inc., Columbia, MD (US)

(21) Appl. No.: 13/146,510

(22) PCT Filed: Jan. 27, 2010
(86) PCT No.: PCT/US10/22257

§ 371 Date: Nov. 9, 2011

Related U.S. Application Data
(60) Provisional application No. 61/147,498, filed on Jan. 27, 2009.

(51) Int. Cl. A61K 31/714; A61P 27/04; A61P 35/00; A61P 31/00; A61P 27/06; A61P 27/02; A61P 29/00

(52) U.S. Cl. 514/52

(57) ABSTRACT

The present invention is directed to methods of treating eye disease. In one embodiment, the method can comprise administering a bioconjugate to a subject to treat the eye disease, where the bioconjugate comprises a taxane covalently bonded to a cobalamin. Additionally, the bioconjugate can be dissolved in an aqueous solution prior to administration.

### BACKGROUND

The efficacy of certain drugs in treating disease is often dependent on their toxicity, biologically availability, or how readily an effective amount of the drug can be delivered to a specific location in a subject's body, particularly to a specific type of tissue or population of cells. Therefore, methods and compositions that lower toxicity, increase bioavailability, or facilitate drug targeting can be of considerable value to the pharmaceutical and medicinal arts. One approach to this need involves using molecules that have generally understood transport mechanisms and which can be induced to release drugs in site-specific fashion.

One such mechanism involves the use of cobalamin (Cbl). Cobalamin is an essential biomolecule, the size of which prevents it from being taken up from the intestine and into cells by simple diffusion, but rather by facultative transport. Cobalamin binds to a specific protein, and the complex may be actively taken up through a receptor-mediated transport mechanism. In the small intestine, cobalamin binds to intrinsic factor (IF) secreted by the gastric lining. The Cbl-IF complex binds to IF receptors on the lumenal surface of cells in the ileum and is transcytosed across these cells into the bloodstream. Once there, cobalamin binds to one of three transcobalamins (TCs) to facilitate its uptake by cells. The receptor-mediated nature of cobalamin uptake imparts a degree of cell-specificity to cobalamin metabolism, in that cobalamin can be absorbed and metabolized by cells that present the correct receptor(s).

Several patents have utilized cobalamin for various purposes. For example, Grissom et al. has obtained several U.S. Pat. Nos. 6,790,827; 6,777,237; and 6,776,976; using organocobalt complexes. Russell-Jones et al. has also utilized cobalamin to increase uptake of active agents, as described in a series of patents, including U.S. Pat. Nos. 5,863,900; 6,159,502; and 5,449,720. In addition to this, research and development for methods and compositions having increased bioavailability of various pharmaceutical agents continue to be sought.

### SUMMARY

It has been recognized that it would be advantageous to develop compositions and methods for delivery of taxanes. Briefly, and in general terms, the invention is directed to methods of treating an eye disease using a taxane bioconjugate. In one embodiment, a bioconjugate is administered to a subject where the bioconjugate comprises a taxane covalently bonded to the 5′-OH of a cobalamin, or more generally, one of the various forms of vitamin B₁₂. In another embodiment, the bonding is through a cleavable linker and one or more optional spacers. In another embodiment, a cobalamin-taxane bioconjugate can be present in an aqueous solution, and can have a water solubility of at least 50 mg/ml, or even at least 100 mg/ml. Methods of administering and/or treating an eye disease include administering a cobalamin-taxane conjugate as an intra-ocular, oral, parenteral, or dermal composition.

Additional features and advantages of the invention will be apparent from the detailed description which follows, taken in conjunction with the accompanying drawings, which together illustrate, by way of example, features of the invention.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 depicts a bioconjugate in accordance with an embodiment of the invention;

FIG. 2A shows the first part an exemplary process for synthesis of the bioconjugate of FIG. 1;

FIG. 2B shows the second part an exemplary process for synthesis of the bioconjugate of FIG. 1;

FIG. 3 shows an exemplary process for synthesizing a bioconjugate in accordance with another embodiment of the invention;

FIGS. 4 through 9 each show the results of high performance liquid chromatography (HPLC) analyses of products of the synthesis process in FIGS. 2A and 2B; and

FIGS. 10 through 12 each show the results of HPLC analyses of products of the synthesis process in FIG. 3; and

FIG. 13 is a graph showing the effects of treatment with a bioconjugate in accordance with an embodiment of the present invention on choroidal neovascularization as compared to controls.

Reference will now be made to the exemplary embodiments illustrated, and specific language will be used herein to describe the same. It will nevertheless be understood that no limitation of the scope of the invention is thereby intended.

### DETAILED DESCRIPTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

In describing and claiming the present invention, the following terminology will be used in accordance with the definitions set forth below.

It must be noted that, as used in this specification and the appended claims, the singular forms “a,” “an,” and, “the” include plural referents unless the context clearly dictates otherwise. Thus, for example, reference to “a taxane” can include one or more of such taxanes, and reference to “the cobalamin” can include reference to one or more cobalamins.

As used herein, the terms “formulation” and “composition” can be used interchangeably and refer to at least one pharmaceutically active agent, such as a taxane covalently bonded to the 5′-OH of a cobalamin with a covalent linkage. The terms “drug,” “active agent,” “bioactive agent,” “pharmaceutically active agent,” and “pharmaceutical,” can also be used interchangeably to refer to an agent or compound that has measurable specified or selected physiological activity when administered to a subject in an effective amount. As used herein, “carrier” or “inert carrier” refers to typical compounds or compositions used to carry drugs, such as polymeric carriers, liquid carriers, or other carrier vehicles with which a bioactive agent may be combined to achieve a specific dosage form. As a general principle, carriers do not substantially react with the bioactive agent in a manner that substantially degrades or otherwise adversely affects the bioactive agent or its therapeutic potential.

As used herein, “administration,” and “administering” refer to the manner in which a drug, formulation, or composition is introduced into the body of a subject. Various art-known routes such as intra-ocular, oral, parenteral, topical, transdermal, and transmucosal can accomplish administration. Thus, an intra-ocular administration can be achieved by dissolving a bioconjugate in water and delivering directly to the eye; e.g. via injection, eye drops, gels, or other topicals.

An oral administration can be achieved by swallowing, chewing, dissolution via adsorption to a solid medium that can be delivered orally, or sucking an oral dosage form comprising active agent(s).

Parenteral administration can be achieved by injecting a drug composition intravenously, intra-arterially, intramuscularly, intrathecally, or subcutaneously, etc. Topical administration may involve applying directly to affected tissue, such as directly to the eye. Transdermal administration can be accomplished by applying, pasting, rolling, attaching, pouring, pressing, rubbing, etc., of a transdermal preparation onto a skin surface. Transmucosal administration may be accomplished by bringing the composition into contact with any accessible mucous membrane for an amount of time sufficient to allow absorption of a therapeutically effective amount of the composition. Examples of transmucosal administration include inserting a suppository into the rectum or vagina; placing a composition on the oral mucosa, such as inside the cheek, on the tongue, or under the tongue; or inhaling a vapor, mist, or aerosol into the nasal passage. These and additional methods of administration are well known in the art.

The term “effective amount,” refers to an amount of an ingredient which, when included in a composition, is sufficient to achieve an intended compositional or physiological effect. Thus, a “therapeutically effective amount” refers to a non-lethal amount of an active agent sufficient to achieve therapeutic results in treating a condition for which the active agent is known or taught herein to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. In some instances, a “therapeutically effective amount” of a drug can achieve a therapeutic effect that is measurable by the subject receiving the drug. For example, in metronomic dosing, “the “therapeutic effective amount” may increase or decrease during the therapeutic treatment due to inherent genetic variation. The determination of an effective amount is well within the ordinary skill in the art of pharmaceutical, medicinal, and health sciences.

As used herein, “treat,” “treatment,” or “treating” refers to the process or result of giving medical aid to a subject, where the medical aid can counteract a malady, a symptom thereof, or other related adverse physiological manifestation. Additionally, these terms can refer to the administration or application of remedies to a patient or for a disease or injury; such as a medicine or a therapy. Accordingly, the substance or remedy so applied, such as the process of providing procedures or applications, are intended to relieve illness or injury. As used herein, “reduce” or “reducing” refers to the process of decreasing, diminishing, or lessening, as in extent, amount, or degree of that which is reduced. Additionally, the use of the term can include from any minimal decrease to absolute abolishment of a physiological process or effect.

As used herein, “subject” refers to an animal, such as a mammal, that may benefit from the administration of a bioconjugate compound of the present disclosure, including formulations or compositions that include the compound.

As used herein, the term “taxane” generally refers to a class of diterpenes produced by the plants of the genus Taxus (yews). This term also includes those taxanes that have been artificially synthesized. For example, this term includes paclitaxel and docetaxel, and derivatives thereof.

As used herein, the term “cobalamin” refers to an organocobalt complex having the essential structure shown below:

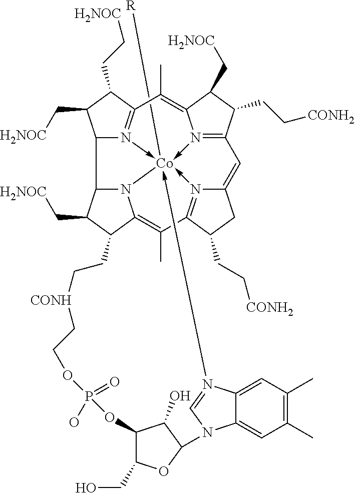

as well as derivatives of this structure in which R may be —CH₃ (methylcobalamin), —CN (cyanocobalamin), —OH (hydroxycobalamin), —C₁₀H₁₂N₅O₃ (deoxyadenosylcobalamin), or synthetic complexes that include a corrin ring and are recognized by cobalamin transport proteins, receptors, and enzymes. The term also encompasses vitamin B₁₂, aquocobalamin, adenosylcobalamin, cyanocobalamin carbanalide, desdimethyl cobalamin, monoethylamide cobalamin, methylamide cobalamin, coenzyme B₁₂, cobamamide derivatives, chlorocobalamin, sulfitocobalamin, nitrocobalamin, thiocyanatocobalamin, benzimidazole derivatives such as 5,6-dichlorobenzimidazole, 5-hydroxybenzimidazole, trimethylbenzimidazole, as well as adenosylcyanocobalamin ((Ade)CN-Cbl), cobalamin lactone, cobalamin lactam and the anilide, ethylamide, monocarboxylic, dicarboxylic and tricarboxylic acid derivatives of vitamin B₁₂, proprionamide derivatives, 5-o-methylbenzylcobalmin, and analogues thereof wherein the cobalt is replaced by another metal atom such as zinc or nickel. The corrin ring of vitamin B₁₂ or its analogues may also be substituted with any substituent which does not completely eliminate its binding to transcobalamin. The term “organocobalt complex” refers to an organic complex containing a cobalt atom having bound thereto 4-5 calcogens as part of a multiple unsaturated heterocyclic ring system, particularly any such complex that includes a corrin ring.

The organocobalt molecule cobalamin is an essential biomolecule with a stable metal-carbon bond. Among other things, cobalamin plays a role in the folate-dependent synthesis of thymidine, an essential building block of DNA. Because cobalamin is a large molecule, cellular uptake of cobalamin is achieved by receptor-mediated endocytosis. The density of receptors in a cell may be modulated in accordance with the cell's need for cobalamin at a given time. For example, a cell may upregulate its expression of cobalamin receptors during periods of high demand for cobalamin. One such time is when the cell replicates its DNA in preparation for mitosis or meiosis. One result of this facultative upregulation is that cobalamin uptake will be higher in cell populations undergoing rapid proliferation than in slower-growing cell populations. This non-uniform uptake profile makes it possible to target delivery of a bioactive agent to high-demand cell populations by linking the agent to cobalamin.

Cobalamin is the most chemically complex of the vitamins. The core structure of the cobalamin molecule is a corrin ring including four pyrrole subunits, two of which are directly connected with the remainder connected through a methylene group. Each pyrrole has a proprionamide substituent that extends radially from the ring. At the center of the ring is a cobalt atom in an octahedral environment that is coordinated to the four corrin ring nitrogens, as well as the nitrogen of a dimethylbenzimidazole group.

The sixth coordination partner can vary as previously discussed; represented by R in the above-defined formula. Six propionamide groups extend from the outer edge of the ring, while a seventh links the dimethylbenzimidazole group to the ring through a phosphate group and a ribose group.

The term “vitamin B₁₂” or “B₁₂” or “VB” or “VB12” has been generally used in two different ways in the art. In a broad sense, it has been used interchangeably with four common cobalamins: cyanocobalamin, hydroxycobalamin, methylcobalamin, and adenosylcobalamin. In a more specific way, this term refers to only one of these forms, cyanocobalamin, which is the principal B₁₂ form used for foods and in nutritional supplements. For the purposes of this invention, this term includes cyanocobalamin, hydroxycobalamin, methylcobalamin, and adenosylcobalamin, unless the context dictates otherwise.

As used herein, the term “bioconjugate” refers to a molecule containing a taxane covalently bonded to cobalamin, e.g., to the 5′-OH atom or by some other linkage mechanism.

Exemplary of the bioconjugate function is the ability to solubilize the taxane upon conjugation. As such, the present bioconjugates can have water solubility allowing for direct dissolution of the bioconjugate in water without the need for solubilization excipients. For example, a taxane can be solubilized with CREMOPHOR®; however, such a solution is toxic, which limits its therapeutic effectiveness and administration. However, the present bioconjugates allow solubilization of taxanes in water, or other aqueous solutions, without the need for further excipients (though the use of other excipients is not precluded), which decreases toxicity and allows for intra-ocular delivery.

Additionally, in one embodiment, the bioconjugate function can serve as a targeted delivery system where the agent or compound to be delivered may be conjugated or otherwise attached to cobalamin without affecting the cobalamin's ability to bind to the appropriate receptor(s). Therefore, it is often the case that the receptor-binding domain(s) of the cobalamin are not modified. Likewise, for successful targeted delivery, the agent or compound can be released from the cobalamin in a therapeutically effective form and at the right location. Some event, substance, or condition can be present in the targeted location that will cause the agent to separate from the carrier. Successful methods of drug targeting can involve agent-cobalamin linkages that are sensitive to particular conditions or processes that are prevalent in the target location.

As used herein, the term “covalent linkage” or “covalent bond” refers to an atom or molecule which covalently or coordinate covalently binds together two components. With regard to the present disclosure, a covalent linkage is intended to include atoms and molecules which can be used to covalently bind a taxane to cobalamin, either directly or through a linker and optionally through one or more spacers. Though not excluded, in one embodiment, the covalent linkage does not prevent the binding of cobalamin to its transport proteins, either by sterically hindering interaction between cobalamin and the protein, or by altering the binding domain of cobalamin in such a way as to render it conformationally incompatible with the protein. Likewise, the covalent linkage should not act in these ways to significantly prevent the binding of the cobalamin-transport protein complex with cobalamin receptors.

As used herein, the term “angiogenesis” or “angiogenic” refers to a physiological process involving the growth of new blood vessels. The growth of new blood vessels is an important natural process occurring in the body, both in health and in disease. In regards to certain eye diseases, the term “anti-angiogenic” refers to those compounds or agents that inhibit the growth of new blood vessels, effectively cutting off the existing blood supply of the disease(s). For example, such anti-angiogenic compounds include, but are not limited to, bevacizumab, suramin, sunitinib, thalidomide, tamoxifen, vatalinib, cilenigtide, celecoxib, erlotinib, lenalidomide, ranibizumab, pegaptanib, sorafenib, and mixtures thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the indicated range. Thus, included in this numerical range are individual values such as 2, 3.5, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

In accordance with these definitions, the present invention provides methods of treating eye diseases with compositions in which a taxane or derivative can be covalently bound to a cobalamin. It is noted that when discussing a cobalamin-taxane bioconjugate containing composition or a method of administering such a composition, each of these discussions can be considered applicable to other embodiments describe herein, whether or not they are explicitly discussed in the context of that embodiment. Thus, for example, in discussing taxanes used in compositions having bioconjugates, those taxanes can also be used in the method for administering such bioconjugate compositions, and vice versa.

In one embodiment, a method of treating an eye disease can comprise administering a bioconjugate to a subject to treat the eye disease. The bioconjugate can comprise a taxane covalently bonded to a cobalamin. In one particular embodiment, the taxane is covalently bonded to the 5′-OH of the cobalamin, and in another embodiment, the bonding occurs through a cleavable linker and one or more optional spacers. In yet another embodiment, the bioconjugate is present as a solubilized compound in an aqueous solution. The step of administering can be accomplished by various methods as are known in the art.

In one embodiment, the step of administering can be by intra-ocular administration or delivery. In another embodiment, the step of administering can be by oral administration or delivery. In yet another embodiment, the step of administering can be by parenteral administration or delivery. In still yet another embodiment, the step of administering can be by topical delivery to the tissue site, or by dermal or mucosal administration or delivery.

The methods of the present invention can be used to treat eye diseases in general, and in one embodiment, eye diseases that can benefit from anti-angiogenic activity. As such, the eye disease can be at least one of age-related macular degeneration, proliferative diabetic retinopathy, non-proliferative diabetic retinopathy, retinopathy of prematurity, corneal graft rejection, neovascular glaucoma, rubeosis, pterygia, abnormal blood vessel growth of the eye, uveitis, dry-eye syndrome, post-surgical inflammation and infection of the anterior and posterior segments, angle-closure glaucoma, open-angle glaucoma, post-surgical glaucoma procedures, exopthalmos, scleritis, episcleritis, Grave's disease, pseudotumor of the orbit, tumors of the orbit, orbital cellulitis, blepharitis, intraocular tumors, retinal fibrosis, vitreous substitute and vitreous replacement, iris neovascularization from cataract surgery, macular edema in central retinal vein occlusion, cellular transplantation (as in retinal pigment cell transplantation), cystiod macular edema, psaudophakic cystoid macular edema, diabetic macular edema, pre-phthisical ocular hypotomy, proliferative vitreoretinopathy, extensive exudative retinal detachment (Coat's disease), diabetic retinal edema, diffuse diabetic macular edema, ischemic opthalmopathy, pars plana vitrectomy (for proliferative diabetic retinopathy), pars plana vitrectomy for proliferative vitreoretinopathy, sympathetic ophthalmia, intermediate uveitis, chronic uveitis, retrolental fibroplasia, fibroproliferative eye diseases, acquired and hereditary ocular conditions such as Tay-Sach's disease, Niemann-Pick's disease, cystinosis, and/or corneal dystrophies.

In one specific embodiment, the present bioconjugates can treat age-related macular degeneration (AMD). Specifically, AMD general can be described in two forms: dry and wet. Dry is most common and does not have neovascularization. However, dry AMD can lead to wet AMD. Wet AMD has neovascularization which is the development of abnormal leaky blood vessels in the macular of the eye. This can result in blindness and/or very impaired vision. Wet AMD is an angiogenic process, i.e., it is the development of new blood vessels that are weak and leaky. These occur in the macula and as a result, can also lead to bleeding in the eyes from the vessels leaking blood. As such, the present bioconjugates can be used for the treatment of AMD, as a result of their anti-angiogenic benefits, as further described herein. Additionally, in another embodiment, the present bioconjugates can treat diabetic retinopathy (both non proliferative and proliferative) as such diseases are known to have abnormal blood vessel growth.

The present eye diseases can benefit from administration of the present bioconjugates, e.g., B₁₂-paclitaxel, since such bioconjugates are water soluble allowing for direct solubilization in water, or other aqueous solutions, without the need for toxic solubilizing excipients, e.g., CREMOPHOR®. Additionally, the bioconjugates can be nontoxic in the eye at doses up to 85 μg/2 μL.

It has been recognized that the attachment of a taxane to a cobalamin can significantly increase the water solubility of the taxane as a cobalamin-taxane bioconjugate. Thus, such an arrangement can be beneficial for treating eye disease, though other forms of such bioconjugates can also be used when solubility is not the objective, e.g., emulsions, microemulsions, liposomes, etc.

Generally, taxanes are insoluble in water. For example, paclitaxel has a water solubility of less than 0.004 mg/ml. However, when conjugated to the 5′-OH of a cobalamin, as shown in the structures described herein, a cobalamin-paclitaxel bioconjugate can exhibit significant water solubility so as to make delivery in an aqueous formulation possible. For example, in one embodiment, a cobalamin-taxane bioconjugate can have a water solubility of at least 0.5 mg/ml. In another embodiment, a cobalamin-taxane bioconjugate can have a water solubility of at least 10 mg/ml. In yet another embodiment, the water solubility can be at least 50 mg/ml. In still yet another embodiment, the water solubility can be at least 100 mg/ml. As such, the cobalamin-taxane bioconjugates provided herein can be orally administered to a subject. In one embodiment, the cobalamin-taxane bioconjugate can have at least a 10 fold increase in water solubility compared to the unconjugated taxane. In another embodiment, the increase can be at least 100 fold. In yet another embodiment, the increase can be at least 1000 fold.

Additionally, it has been recognized that the cobalamin-taxane bioconjugates disclosed herein can have increased bioavailability in a subject. Bioavailability of a compound can be dependent on P-Glycoprotein (P-gp), an ATP-dependent drug pump, which can transport a broad range of hydrophobic compounds out of a cell. This can lead to the phenomenon of multi-drug resistance. Expression of P-gp can be quite variable in humans. Generally, the highest levels can be found in the apical membranes of the blood-brain/testes barrier, intestines, liver, and kidney. Over-expression in patients can undermine treatment as the drug is pumped out via this pump. P-gp can also affect the penetration of the drug to solid tumors or other maladies. P-gp has been shown to affect the ability of taxanes, such as paclitaxel or docetaxel, to enter the cells and become bioavailable. Therefore, the bioconjugates of the present invention can be structurally different as to bypass the P-gp pathway leading to increased bioavailability of the bioconjugate. Additionally, cobalamin bioconjugates can use a facultative transport mechanism, which would also bypass the P-gp pathway leading to increased bioavailability.

The present disclosure also relates to solubilization and drug delivery of taxanes and their derivatives for the treatment of the eye via a cobalamin-taxane bioconjugate, e.g., oral, parenteral, topical, ocular, etc. In addition, it is noted that there may be an inherent targeting effect via the cobalamin molecule. When introduced into the bloodstream or gastrointestinal tract of a subject, such a bioconjugate can take advantage of existing systems for absorption, transport, and binding of cobalamin. In this way, the taxane can be transported to cells that bear receptors for cobalamin and be taken up by those cells. As noted above, some cells or cell populations in a given is subject can utilize cobalamin more heavily at a given time than other cells; consequently expression of cobalamin receptors is upregulated in such cells at those times. Thus, when the bioconjugate is administered to a subject, more of the taxane can be taken up by these cells than by other cells. Thus, the present invention provides a method for concentrating a taxane to sites where cells are utilizing cobalamin heavily. Increased demand for cobalamin is associated with, among other things, rapid cellular proliferation. Therefore, the present invention can be used to concentrate taxanes in neoplastic cells in a subject suffering from a proliferative disease.

In accordance with an embodiment of the present invention, a bioconjugate to be used for treating an eye disease can comprise a cobalamin or a cobalamin derivative; a linker covalently bound to the 5′-OH moiety of the cobalamin or cobalamin derivative; and a taxane covalently bound to the linker. In a particular embodiment, the taxane is cleavable from the linker and/or the linker is cleavable from cobalamin by an intracellular enzyme. In a more detailed embodiment, the bioconjugate can have general Formula I, as follows:

VB-(SPa)ₙ-CL-(SPb)ₘ-DG Formula I

wherein:

a. CL is a linker that is cleavable from the VB, SPa, SPb and/or DG by way of intracellular enzyme;

b. VB is cobalamin, or a derivative or analogue thereof, covalently bound to CL and SPa, if present, via the 5′-OH group of the ribose ring of VB;

c. SPa and SPb are optional spacers independently selected at each occurrence from the group consisting of a covalent bond, divalent functional group, or non-peptide residue, wherein SPa and SPb can be located on either side of CL; and

d. DG is a taxane,

The values n and m can be independently selected at each occurrence from 0, 1, 2, or 3. In one embodiment, the conjugate optionally possesses one or more is protecting groups.

A spacer is optional in the compound of Formula I. Zero, one or two spacers or a combination of spacers can be included. The spacer serves to adjust the distance between the cobalamin and linker, cobalamin and drug, or linker and drug. The distance from the 5′-OH of cobalamin to the point of attachment of the drug to the CL or spacer is sufficient to permit binding of transcobalamin and of an enzyme responsible for cleaving the conjugate. Depending upon the drug being used and the particular form of cobalamin being used, the distance may vary for optimal performance.

Spacers can also be introduced either to improve the transcobalamin affinity of the conjugate or to overcome problems in the coupling of the cobalamin, linker and/or the drug arising from unfavorable steric interactions or to increase the bioactivity of the drug in the conjugate. The spacer compounds may also act as linking agents, being bi-functional compounds with selected functional groups on each end to react with suitable functional groups located on the linker or the cobalamin.

Since the spacers are optional, specific embodiments of the conjugate include: VB-(Spa)ₚ-CL-DG (Formula II), VB-CL-(SPb)_{q}-DG (Formula III), VB-CL-DG (Formula IV), VB-CL-(SPa)ₚ-(SPb)_{q}-DG (Formula V), VB-(SPa)ₚ-(SPb)_{q}-CL-DG (Formula VI), and VB-(SPa)²(SPa)¹-CL-(SPb)¹(SPb)²-DG (Formula VII), wherein “p” and “q” are independently selected at each occurrence from 1, 2, or 3.

The spacer SPa or SPb can comprise optionally substituted saturated or unsaturated, branched or linear, C₁₋₅₀ alkylene, cycloalkylene or aromatic groups, optionally with one or more carbons within the chain being replaced with N, O or S, and wherein the optional substituents are selected from, for example, carbonyl, carboxy, hydroxy, amino and other groups. When two spacers are included in the conjugate, they are different in structure. A spacer is adapted to cleave from the anti-tumor drug after the CL is cleaved in the target tissue, thereby releasing the drug intracellularly in a therapeutically effective form. These spacers are designed to allow an intracellular enzyme to approach and cleave the linker. They are also designed to cleave from the drug to form the active form of the drug after the linker has been cleaved. A spacer is covalently bound to the CL, DG and VB such that it is sufficiently chemically stable to remain bound thereto until the conjugate is delivered to a target cell or tissue. In a specific embodiment, the spacer is cleaved intracellularly, either by an enzyme or other means, within a target cell or tissue. If a spacer is cleavable, it can be cleaved by the same or a different means as a cleavable linker to which it is attached. Alternatively, the spacer will substantially cleave itself from the cleavable linker and/or drug after the cleavable linker is cleaved intracellularly from VB or SPa. In a specific embodiment, an intracellular enzyme initially releases CL-SPb-DG (or CL-DG) from VB-SPa or VB. The remaining residue CL-SPb-DG (or CL-DG) then cleaves by itself thereby releasing free drug intracellularly. Cleavage need not be solely enzymatic, as it can include additional chemical cleavage provided enzymatic cleavage occurs first.

When the spacer is a divalent functional group it can be attached to the cobalamin, cleavable linker or drug in a forward or reverse direction. Suitable divalent functional groups include —NHNH—, —NH—, —O—, —S—, —SS—, —CH₂—, —NHCO—, —CONH—, —CONHNHCO—, —N═N—, —N═CH—, —NHCH₂—, —NHN═CH—, —NHNHCH₂—, —SCH₂—, —CH₂S—, —NHC═ONH—, —NHC═SNH—, —NHC═NHNH—, —COO—, and —OCO—.

The cleavable linker “CL” is intended to resist breakdown from enzymes in the plasma and optionally gastrointestinal tract of a mammal. In a particular embodiment, the cleavable linker undergoes intracellular cleavage after it is taken up by a cell. The CL can be a peptide or non-peptide.

The combination of elements (SPa)ₙ-CL-(SPb)ₘ of the conjugate, and other embodiments thereof as described herein, together form a “conjugating unit” having a structure as defined by the specific definition of the individual elements SPa, SPb, and CL and the variables n and m. In other words, the “conjugating unit” will be defined by any permissible embodiment of (SPa)ₙ-CL-(SPb)ₘ.

According to a specific embodiment, the conjugating unit of the present invention is made up of a carboxylic acyl unit, and a protein peptide sequence. It may also contain a self-immolating spacer that spaces the drug and the protein peptide sequence.

In a specific embodiment of the conjugate, the conjugating unit is defined as “A-Y-Z-X-W” (Formula VIII) in which “A” is a “carboxylic acyl unit”, “Y” and “Z” are each amino acids and together form the protein peptide sequence, and “X” and “W” are individually self-immolating spacers that space the protein peptide and the drug. The conjugating unit A-Y-Z-X-W is a subset of the conjugating unit (SPa)ₙ-CL-(SPb)ₘ and the conjugating unit (VB-(Spa)²(Spa)¹-CL-(SPb)¹(SPb)²-DG).

Specific embodiments include those wherein:

Y is at least one amino acid selected from the group consisting of alanine, valine, leucine, isoleucine, methionine, phenylalanine, tryptophan and proline, preferably phenylalanine or valine; and

Z is at least one amino acid selected from the group consisting of lysine, lysine protected with acetyl or formyl, arginine, arginine protected with tosyl or nitro groups, histidine, ornithine, ornithine protected with acetyl or formyl, and citrulline, preferably lysine, or citrulline.

In a specific embodiment, the peptide sequence is tailored so that it can be selectively enzymatically cleaved from the conjugate by one or more proteases in a target cell. The chain length of protein peptide sequence generally ranges from that of a dipeptide to that of a tetrapeptide. However, a protein peptide sequence as long as eight amino acid residues may also be employed.

Suitable exemplary peptide linker groups include by way of example and without limitation include Phe-Lys, Val-Lys, Phe-Phe-Lys, D-Phe-L-Phe-Lys, Gly-Phe-Lys, Ala-Lys, Val-Cit, Phe-Cit, Leu-Cit, Ile-Cit, Trp-Cit, Phe-Ala, Gly-Phe-Leu-Gly, Ala-Leu-Ala-Leu, Phe-N⁹-tosyl-Arg, and Phe-N⁹-Nitro-Arg.

Other linkages that will serve the functions described above will be known to those having skill in the art, and are encompassed by the present invention. Other possible linkers, spacers, and enzymes for targeting such linkages are described in U.S. Pat. No. 7,232,805 to Weinshenker et al. which is incorporated herein by reference in its entirety.

Such a linkage can serve as a target for an enzyme that will cleave the linkage, releasing the taxane from the cobalamin. Such an enzyme can be present in the subject's bloodstream and thereby release the taxane into the general circulation, or it can be localized specifically to a site or cell type that is the intended target for delivery of the taxane. Alternatively, the linkage can be of a type that will cleave or degrade when exposed to a certain environment or, particularly, a characteristic of that environment such as a certain pH range or range of temperatures. The linkage can be of a “self-destructing” or a “self-immolative” type, i.e. it will be consumed in the process of cleavage, so that said cleavage will yield only the original cobalamin and the taxane molecules absent any remaining large sections of the linkage. Those having skill in the art will recognize other release mechanisms derived from various linkages that can be used in accordance with the present invention.

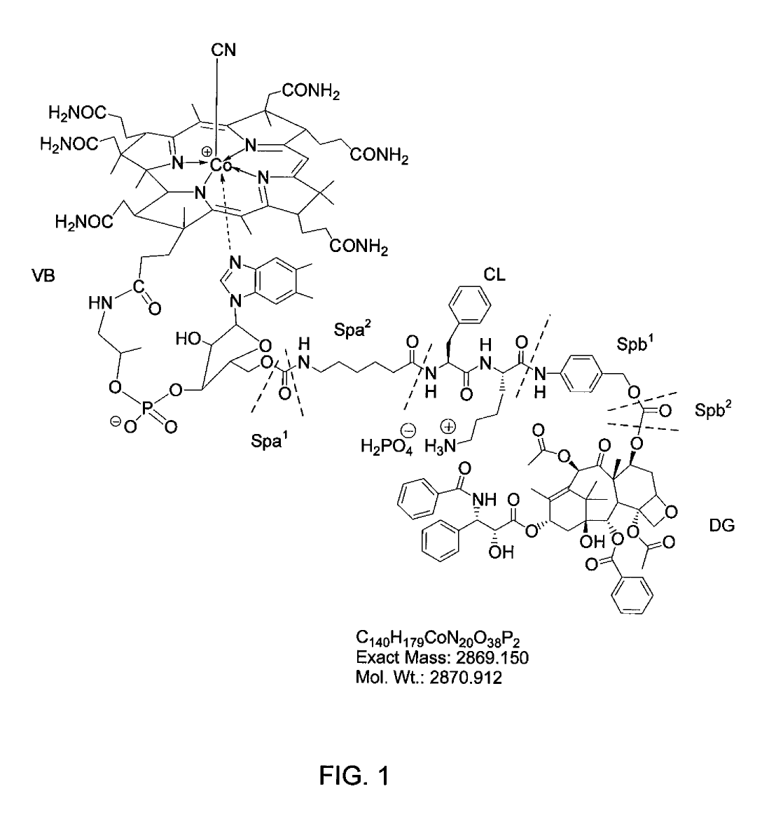

FIG. 1 depicts an exemplary conjugate according to the present invention. The conjugate, which can be made according to the process of Example 1, comprises taxol bound to a spacer (Spb), which is bound to a dipeptide cleavable linker (CL) that is then bound to a second spacer (Spa) which is finally bound to the 5′-OH moiety of the ribose ring of cobalamin (VB). Each spacer comprises two spacer residues (e.g. Spa¹ and Spa²). Accordingly, the compound depicted has the following formula: VB-(SPa)²(SPa)¹-CL-(SPb)¹(SPb)²-DG (Formula VII).

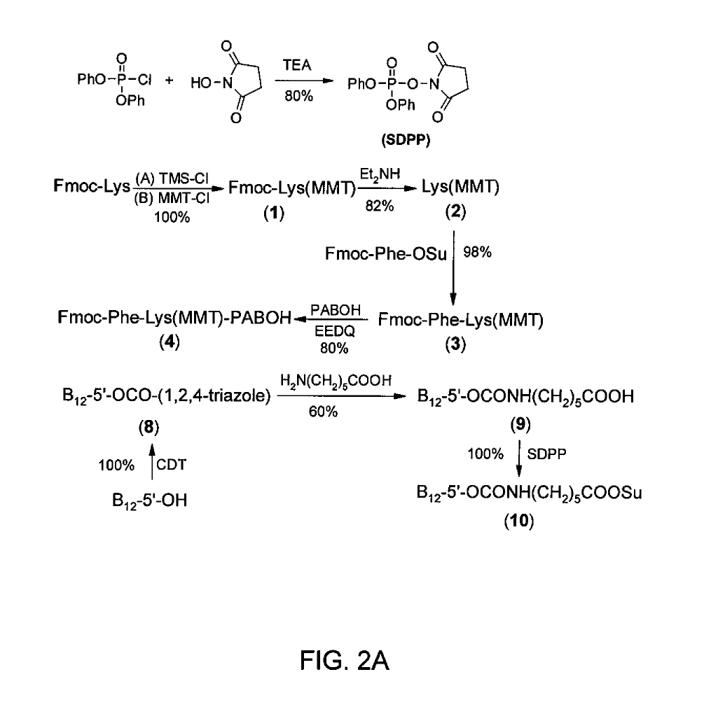

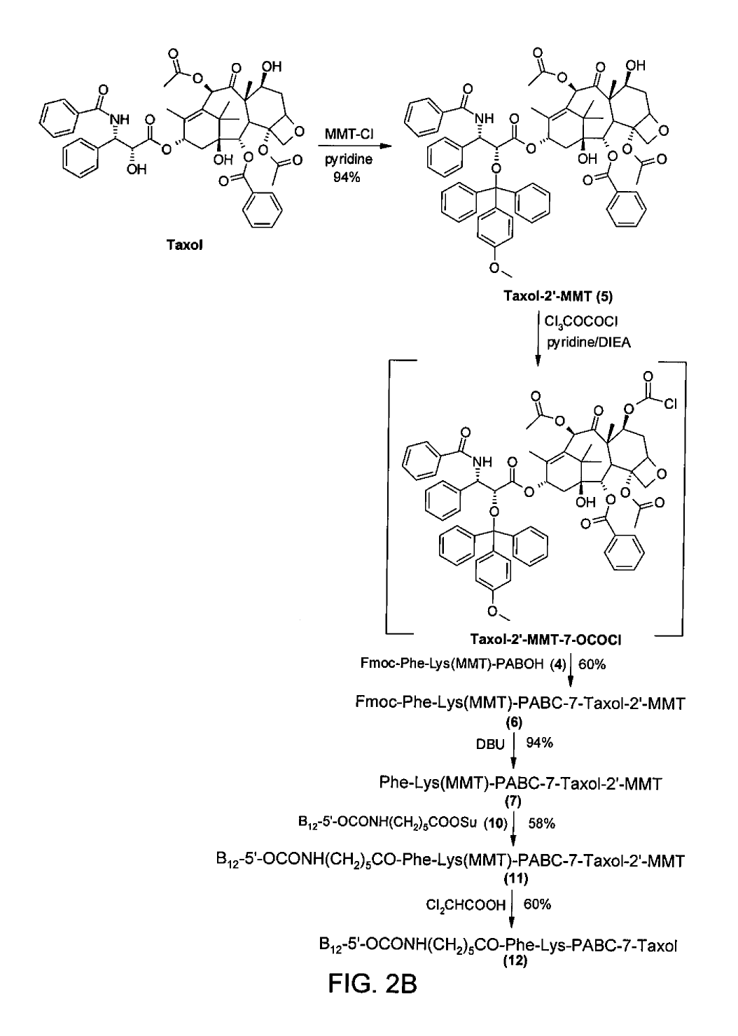

An exemplary synthetic process for the depicted conjugate is detailed in FIGS. 2A and 2B and further in Example 1. The first part of the synthesis, depicted in FIG. 2A, concerns preparation of the cleavable linker Phe-Lys. The second part of the synthesis, also shown in FIG. 2A, concerns attachment of the cleavable linker to the first spacer (PABC). Another step shown in FIG. 2A involves attaching the second spacer to cobalamin. FIG. 2B shows the addition of a protecting group (MMT) to taxol.

The further steps shown involve attaching the protected taxol to the first spacer-linker group and then attachment of this complex to the cobalamin and second spacer.

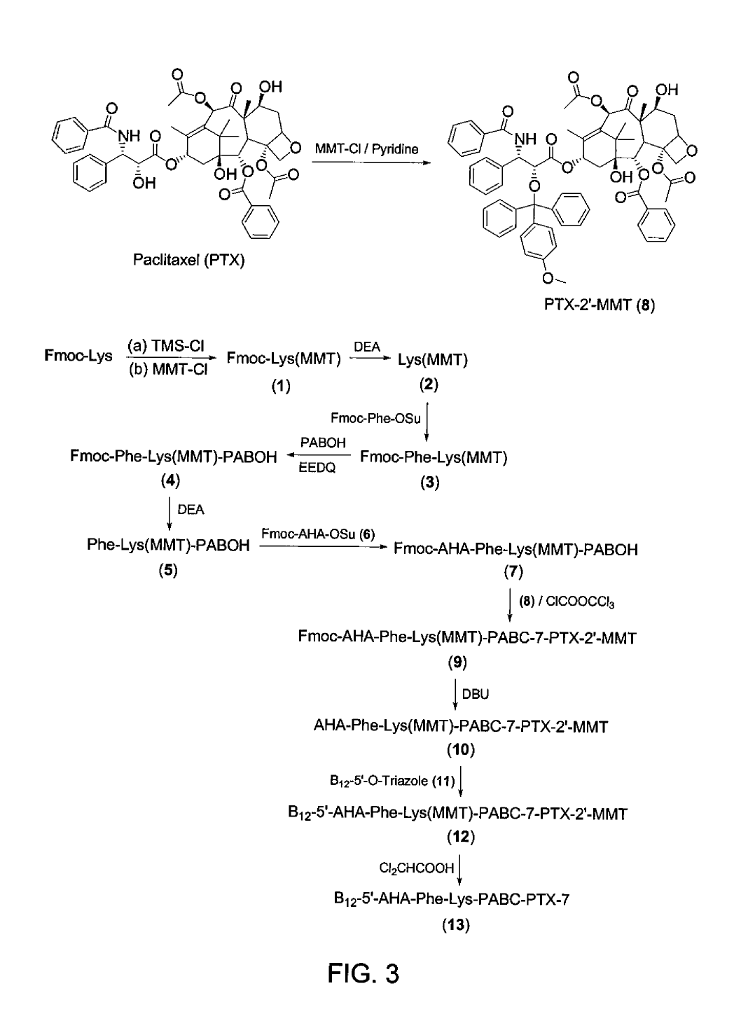

FIG. 3 shows an exemplary process for making another conjugate in accordance with an embodiment of the invention. In this process, a protecting group (MMT) is added to paclitaxel (Taxol). The synthesis proceeds with preparation of the cleavable linker Phe-Lys, followed by and the addition of a first and second spacer to either end of the linker. Then the paclitaxel and cobalamin are each attached to the conjugating unit. The synthesis is detailed further in Example 2 below.

Again, though specific compounds are shown by way of example, it is understood that many different combinations of taxanes and cobalamin can be prepared in accordance with embodiments of the present disclosure. For example, the taxane for use can be selected from the group consisting of paclitaxel and docetaxel, derivatives thereof, and mixtures thereof. In one embodiment, the taxane can be paclitaxel. In another embodiment, the taxane can be docetaxel. The cobalamin can be selected from the group consisting of cyanocobalamin including anilide, ethylamide, proprionamide, monocarboxylic, dicarboxylic, and tricarboxylic acid derivatives thereof; hydroxycobalamin including anilide, ethylamide, proprionamide, monocarboxylic, dicarboxylic, and tricarboxylic acid derivatives thereof; methylcobalamin including anilide, ethylamide, proprionamide, monocarboxylic, dicarboxylic, and tricarboxylic acid derivatives thereof; adenosylcobalamin including anilide, ethylamide, proprionamide, monocarboxylic, dicarboxylic, and tricarboxylic acid derivatives thereof; aquocobalamin; cyanocobalamin carbanalide; desdimethyl cobalamin; monoethylamide cobalamin; methlyamide cobalamin; 5′-deoxyadenosylcobalamin; cobamamide derivatives; chlorocobalamin; sulfitocobalamin; nitrocobalamin; thiocyanatocobalamin; benzimidazole derivatives including 5,6-dichlorobenzimidazole, 5-hydroxybenzimidazole, trimethylbenzimidazole, as well as adenosylcyanocobalamin; cobalamin lactone; cobalamin lactam; 5-o-methylbenzylcobalamin; derivatives thereof; mixtures thereof; and analogues thereof wherein the cobalt is replaced by another metal. In one embodiment, the cobalamin can be one of the vitamin B₁₂ types of cobalamin, and in one specific embodiment, hydroxycobalamin.

The compounds of the present invention can be administered as pharmaceutical compositions in treating various eye diseases. Notwithstanding the ability to solubilize taxanes without the need for solubilizing excipients and/or additives, such a composition can further comprise one or more excipients, including binders, fillers, lubricants, disintegrants, flavoring agents, coloring agents, sweeteners, thickeners, coatings, and combinations thereof. The composition of the present invention can be formulated into a number of dosage forms including syrups, elixirs, solutions, suspensions, emulsions, capsules, tablets, lozenges, and suppositories. Differing administration regimens will call for different dosage forms, depending on factors such as the subject's age, medical condition, level of need for treatment, as well as the desired time course of therapeutic effect. Those having skill in the art will recognize that various classes of excipients can each provide different characteristics to a pharmaceutical composition and that they can be combined in certain ways in accordance with the present invention to achieve an appropriate dosage form.

One aspect of the present invention is that administering the bioconjugate can be more effective in treating an eye disease than administering the taxane and the cobalamin as separate molecules. In light of the fact that taxanes alone can provide anti-angiogenic effects, the present invention provides cobalamin-taxane bioconjugates as anti-angiogenic compounds for treating various eye diseases. The amount of taxane to cobalamin can generally be equal, e.g., the taxane to cobalamin molar ratio can about 1:1. However, the composition can have an excess of cobalamin or taxane that is not covalently bonded. In one embodiment, a composition can have a cobalamin to cobalamin-taxane bioconjugate molar ratio from about 1:2 to about 10:1, or in another embodiment, from about 1.2:1 to about 10:1. Additionally, the bioconjugate can further include additional anti-angiogenic compounds. Such additional anti-angiogenic compounds include, but are not limited to, bevacizumab, suramin, sunitinib, thalidomide, tamoxifen, vatalinib, cilenigtide, celecoxib, erlotinib, lenalidomide, ranibizumab, pegaptanib, sorafenib, and mixtures thereof.

As previously discussed, the bioconjugates of the present invention are readily soluble in water and can be administered to a subject having various eye diseases. As such, the administering can be therapeutically effective while providing low serum levels in the patient, enabling effective treatments having no or very little toxicity. Specifically, the serum levels can be less than 0.01 ng/ml. In another embodiment, the serum levels can be less than 0.001 ng/ml. The taxane of the bioconjugate can be administered at, or equivalent to, about 0.001 μg/day to about 10 μg/day.

As cobalamin receptors are highly upregulated in rapidly proliferating cells as dividing cells require cobalamin for thymidine synthesis in DNA replication. This makes cobalamin a useful vehicle to preferentially deliver drugs to proliferating cells. In one embodiment, administering the bioconjugates of the present invention can be used to achieve serum levels in a subject of about 0.1 ng/ml to about 20,000 ng/ml. Further, the taxanes of the cobalamin-taxane bioconjugates of the present invention can be administered at about 1 mg/kg/day to about 10 mg/kg/day. In one embodiment, the rate can be about 2 mg/kg/day to about 6 mg/kg/day.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

### EXAMPLES

The following provides examples of oral taxanes in accordance with the compositions and methods previously disclosed. Additionally, some of the examples include studies performed showing the effects of oral taxanes on animals in accordance with embodiments of the present invention.

In the following examples, some of the abbreviations used herein are defined as follows:
AA: amino acid
AHA: 6-aminohexanoyl
B₁₂-5′-OH: cyanocobalamin
CDT: 1,1′-carbonyldi(1,2,4-triazole)
DEA: diethylamine
DIC: diisopropylcarbodiimide
DIEA: diisopropylethylamine
DBU: 1,8-Diazabicyclo[5.4.0]undec-7-ene
DCC: dicyclohexylcarbodiimide
DCU: dicyclohexylurea
DMSO: dimethylsulfoxide
EEDQ: 2-ethoxy-1-ethoxycarbonyl-1,2-dihydroquinoline
Fmoc: 9-fluorenyl methoxycarbonyl
HOSu: N-hydroxysuccinimide
HPLC: high performance liquid chromatography
Lys: lysine
MMT: p-methoxyphenyldiphenylmethyl(monomethoxytrityl)
PABOH: p-aminobenzyl alcohol
PABC: p-aminobenzylcarbonyl
Phe: phenylalanine
SDPP: N-Succinimidyl diphenylphosphate
PTX: paclitaxel
TEA: triethylamine
TMS: trimethylsilyl

### Example 1

### Preparation of Cobalamin-Taxol Bioconjugate

The synthesis process depicted in FIGS. 2A and 2B was carried out as follows:

(a.) A taxol (paclitaxel) was purchased from 21CEC PX Pharm Ltd (UK). Cyanocobalamin was obtained from F. Hoffmann-La Roche AG. Amino acid derivatives and EEDQ were from Novabiochem. Fmoc-Phe-OSu was obtained from Advanced ChemTech. SDPP may be obtained from Digital Specialty Chemicals, Inc. All other chemicals and solvents were from Acros, Aldrich, Sigma, Fluka, Fisher or VWR and used without further purification unless stated otherwise. Silica Gel 60 F₂₅₄ aluminium-backed TLC plates were obtained from VWR (P/N EM-5554-7). A Waters HPLC system including a Delta 600 pump with model 600 controller and a 2996 PDA detector was used for both analytical and preparative work. 50 mM phosphoric acid (adjusted to pH 3.0 with ammonia) (A) and acetonitrile/water (9:1, B) were used as aqueous and organic eluents, respectively, unless stated otherwise. A Waters Delta-Pak C₁₈ 15 μm 100 Å 3.9×300 mm column (P/N WAT011797) and 1 mL/min flow rate were used for analytical work; a Waters Delta-Pak Radial Compression C₁₈ 15 μm 100 Å 25×300 mm column (P/N WAT011797) and 41 mL/min flow rate were used for preparative work. Mass spectra were acquired on an Applied Biosystems API 2000 electrospray mass spectrometer in positive ion mode.

(b.) SDPP was synthesized as follows: To an ice-cooled solution of N-hydroxysuccinimide (1.1538 g, 10.0252 mmol, 1.0 eq) and TEA (1.41 ml, 10.0326 mmol, 1.0 eq) in methylene chloride (6 ml) was added diphenyl chlorophosphate (2.07 ml, 10.0094 mmol, 1.0 eq). The mixture was stirred at room temperature for 1 hr. The white solid was filtered off and washed with methylene chloride (5 ml×3). The filtrate was condensed with rotary evaporator and the residue was triturated with ether (30 ml). The resulting white solid was collected and dissolved in ethyl acetate (100 ml), washed with water (30 ml×3), dried over magnesium sulfate. After removal of solvent, 2.7710 g (79.6%) of white solid was obtained. R_{f}: 0.53 (5% CH₃OH/CH₂Cl₂).

(c.) Fmoc-Phe-OSu was synthesized as follows: To a suspension of Fmoc-Phe (7.7482 g, 0.0200 mol, 1.0 eq) and N-hydroxysuccinimide (2.4182 g, 0.0210 mol, 1.05 eq) in methylene chloride (150 ml) cooled in an ice bath, was added DCC (4.3440 g, 0.0211 mol, 1.05 eq). The mixture was stirred at room temperature overnight. The resulting DCU was removed by filtration and the filtrate was condensed and dried in vacuo to give 10.0798 g of white foam. R_{f}: 0.75 (5% CH₃OH/CH₂Cl₂).

(d.) Fmoc-Lys(MMT) (1) was synthesized as follows: To a stirring suspension of Fmoc-Lys (Novabiochem, 5.1067 g, 13.8618 mmol, 1.0 eq) in methylene chloride (75 ml) at room temperature was added trimethylsilyl chloride (Acros, 3.8 ml, 29.7312 mmol, 2.14 eq). The mixture was refluxed at 50° C. for 1 hr (the appearance of the solid in the reaction mixture changed). Then cooled in an ice bath, DIEA (7.5 ml, 43.0561 mmol, 3.11 eq) was added (the mixture became homogeneous) and followed by p-anisyldiphenylmethyl chloride (Acros, 4.4955 g, 14.5580 mmol, 1.05 eq). The orange-red solution was stirred at RT overnight (20 hrs). After removal of solvent, the residue was partitioned between ethyl acetate (200 ml) and pH5 buffer (0.05M phthalic acid, adjusted with 10N KOH to pH 5.0). The organic phase was washed with more pH5 buffer (50 ml×2), water (50 ml×1), brine (50 ml×2), dried over magnesium sulfate. Removal of solvent and being dried in vacuo gave pale yellow foam (9.7336 g). TLC showed trace of impurities (R_{f}=0.45 for product, by 10% CH₃OH/CHCl₃). ¹H-NMR (CDCl₃, 300 MHz): OK (no TMS group).

(e.) Lys(MMT) (2) was prepared as follows: To a stirring solution of Fmoc-Lys(MMT) (9.7336 g, assuming 13.8618 mmol) in 1:1 CH₂Cl₂/acetonitrile (100 ml) at room temperature was added diethylamine (Acros, 100 ml). The mixture was stirred at RT for 1.5 hrs. After removal of solvent, the residue was flushed with acetonitrile at 60° C. (90 ml×2, being stirred for 5 min), washed with acetonitrile (20 ml×3) and ether (20 ml×3). The solid was then dissolved as far as possible in 1:1 CH₂Cl₂/CH₃OH (200 ml) and some solid byproduct was removed by filtering through filter paper. After removal of solvent and being dried in vacuo, pale yellow foam (4.7707 g, 82.2% based on Fmoc-Lys) was obtained. TLC(R_{f}=0, by 10% CH₃OH/CHCl₃) showed no starting material. ES(+)−MS: 147 (Lys+1), 273 (MMT). ¹H-NMR (DMSO-d6, 300 MHz): OK.

(f.) Fmoc-Phe-Lys(MMT) (3) was synthesized as follows: To a stirring suspension of Fmoc-Phe-OSu (2.0702 g, 4.2728 mmol, 1.0 eq) and Lys(MMT) (1.7995 g, 4.2995 mmol, 1.01 eq) in DMF (30 ml) was added DIEA (1.5 ml, 8.6112 mmol, 2.02 eq). The solid dissolved gradually and the solution was stirred at RT overnight. The reaction mixture was partitioned between ethyl acetate (100 ml) and pH5 buffer (0.05M phthalic acid, adjusted with 10N KOH to pH 5.0, 200 ml). The aqueous solution was extracted with more ethyl acetate (50 ml×2). The combined organic phase was washed with brine (50 ml×3), dried over MgSO₄. After removal of solvent and being dried in vacuo, 3.3014 g (98.1%) of pale-yellow foam was obtained. TLC(R_{f}=0.43, by 10% CH₃OH/CHCl₃) showed a small impurity spot. ES(+)−MS: 788 (M+1), 810 (M+Na), 538 (M−MMT+Na), 273 (MMT). ¹H-NMR (DMSO-d6, 300 MHz): OK.

(g.) Fmoc-Phe-Lys(MMT)-PABOH (4) was synthesized as follows: To a stirring solution of Fmoc-Phe-Lys(MMT) (3.3014 g, 4.1898 mmol, 1.0 eq) and 4-aminobenzyl alcohol (Fluka, 0.6219 g, 5.0495 mmol, 1.21 eq) in CH₂Cl₂ (20 ml) was added 2-ethoxy-1-ethoxycarbonyl-1,2-dihydroquinoline (EEDQ, Novabiochem, 1.5589 g, 6.3037 mmol, 1.50 eq). The mixture was stirred at RT overnight. After removal of solvent, the residue was triturated with ether (50 ml). The mixture was left to stand at RT for 2 hours and then the solid was collected, washed with ether (15 ml×3), dried in vacuo. 2.1071 g (56.3%) of white solid was obtained. The ether filtrate was condensed. The residue was suspended in benzene (10 ml) and precipitated with hexane (10 ml). This process was repeated two more times. The resulting solid was collected, washed with benzene/hexane (1:1, 10 ml×3), dried in vacuo. Another 0.8864 g (23.7%) of white solid was obtained. Total yield: 80.0%. TLC(R_{f}=0.57, by 10% CH₃OH/CHCl₃) showed trace of impurity. ES(+)−MS: 893 (M), 915 (M+Na), 810 (M−PABOH+Na), 273 (MMT). ¹H-NMR (DMSO-d6, 300 MHz): OK. The composition may be purified by silica column (eluting with 5% CH₃OH/CH₂Cl₂ with a few drops of TEA).

(h.) Taxol-2′-MMT (5) was synthesized as follows: To a stirring solution of paclitaxel (1.0033 g, 1.1749 mmol, 1.0 eq) and p-anisylchlorodiphenylmethane (2.8972 g, 9.3821 mmol, 7.98 eq) in CH₂Cl₂ (20 ml) was added pyridine (0.78 ml, 9.5651 mmol, 8.14 eq). The solution was stirred at RT overnight. After removal of solvent, the residue was dissolved in ethyl acetate (200 ml) and cold pH5 buffer (0.05M phthalic acid, adjusted with 10N KOH to pH 5.0, 100 ml). The organic phase was separated and washed with cold pH 5 buffer (100 ml×2), water (100 ml×1) and brine (100 ml×1), dried over MgSO₄. After removal of solvent, the residue was purified by silica column (5×10 cm, packed with 4:1 hexane/ethyl acetate; Sample was dissolved in ethyl acetate, adsorbed to 10 g of silica gel, air-dried and loaded onto the column), eluting with hexane/ethyl acetate (2:3, 550 ml), giving 1.2451 g (94.1%) of white solid. R_{f}: 0.52 (2:3 hexane/ethyl acetate). ES(+)−MS: 1148.2 (M+Na).

(i.) Fmoc-Phe-Lys(MMT)-PABC-7-Taxol-2′-MMT (6) was synthesized as follows: To an ice-cooled solution of Taxol-2′-MMT-7-OH (1.3825 g, 1.1795 mmol, 1.0 eq) in methylene chloride (18 mL) was added DIEA (0.205 ml, 1.1769 mmol, 1.00 eq), pyridine (0.096 ml, 1.1772 mmol, 1.00 eq) and then diphosgene (0.071 ml, 0.5886 mmol, 0.50 eq). The ice bath was removed and the solution was stirred at RT for 2 hours. Then re-cooled in an ice-bath, a solution of Fmoc-Phe-Lys(MMT)-PABOH (1.0540 g, 1.1801 mmol, 1.00 eq) and DIEA (0.205 ml, 1.1769 mmol, 1.00 eq) in methylene chloride (60 ml, due to the low solubility of Fmoc-Phe-Lys(MMT)-PABOH in methylene chloride) was added via a syringe. The solution was stirred at RT overnight. The reaction mixture was condensed to about 10 ml and then diluted with ethyl acetate (200 ml), washed with pH 5 buffer (0.05M phthalic acid, adjusted with 10N KOH to pH 5.0, 100 ml×3), water (100 ml×1) and brine (100 ml×1), dried over MgSO₄. After removal of solvent, the residue was purified by silica column (5×11 cm, packed with 9:1 methylene chloride/ethyl acetate, sample dissolved in 9:1 methylene chloride/ethyl acetate), eluting with methylene chloride/ethyl acetate (3:1, 500 mL), giving 1.4410 g (59.7%) of white solid. R_{f}: 0.62 (75:25 methylene chloride/ethyl acetate).

(j.) Phe-Lys(MMT)-PABC-7-Taxol-2′-MMT (7) was synthesized as follows: To a stirring solution of Fmoc-Phe-Lys(MMT)-PABC-7-Taxol-2′-MMT (1.4410 g, 0.7045 mmol, 1.0 eq) in dry THF (20 ml) was added 1,8-diazabicyclo[5.4.0]undec-7-ene (DBU, 0.215 mL, 1.4264 mmol, 2.02 eq, final concentration: 1%). The solution was stirred at RT for 8 minutes. The reaction mixture was added to stirring hexane (90 mL). The resulting precipitate was collected, washed with hexane (10 mL×3), dried in vacuo, giving 1.2015 g (93.5%) of white solid. R_{f}: 0.4 (5% methanol/methylene chloride). ES(+)−MS: 1278.8 (M−2MMT)⁺; ES(−)−MS: 910.6 (M−2H)²⁻.

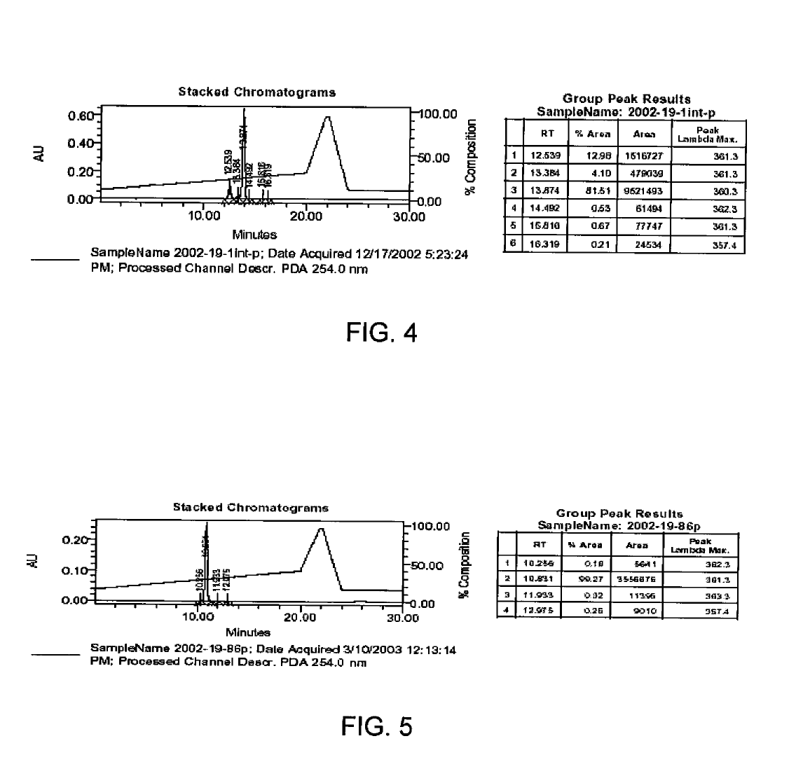

(k.) B₁₂-5′-OCO(1,2,4-triazole) (8) was synthesized as follows: To a stirring solution of DMSO (30 ml) was added cyanocobalamin (2.0380 g, 1.5036 mmol, 1.0 eq) and 1,1′-carbonyldi(1,2,4-triazole) (0.3759 g, 2.2903 mmol, 1.52 eq). The mixture was stirred at RT for 10 min. HPLC indicated about 90% of starting cyanocobalamin was converted to the product. After 30 min, the reaction mixture was added to stirring is mixture of CH₂Cl₂/ether (1:1, 200 ml). The resulting precipitate was collected, washed with acetone (50 ml×3) and ether (50 ml×1), dried in vacuo, giving 2.3706 g of red powder. HPLC: 81.5% pure (gradient: 10-30% B over 20 min) as shown in FIG. 4.

(l.) B₁₂-5′-OCONH(CH₂)₅COOH (9) was synthesized as follows: Compound (8) was added to a stirring suspension of 6-aminohexanoic acid (0.2180 g, 1.6620 mmol, 1.11 eq) and DIEA (0.54 ml, 3.100 mmol, 2.06 eq) in DMSO (30 ml). The mixture was stirred at RT overnight. The reaction mixture was filtered through glass wool to remove the un-reacted 6-aminohexanoic acid. The filtrate was added to a stirring mixture of CH₂Cl₂/ether (1:1, 200 ml). The resulting precipitate was collected, washed with acetone (50 ml×3) and ether (50 ml×1), dried in vacuo, giving 2.3562 g of red powder. It was purified by silica column (5×12 cm), eluting with water (monitor the fractions by HPLC), giving 1.3546 g (59.6%) of red powder. R_{f}: 0.85 (water). ES(+)−MS: 1513.8 (M+1). HPLC indicated about 99% pure (gradient: 15-40% B over 20 min) as shown in FIG. 5.

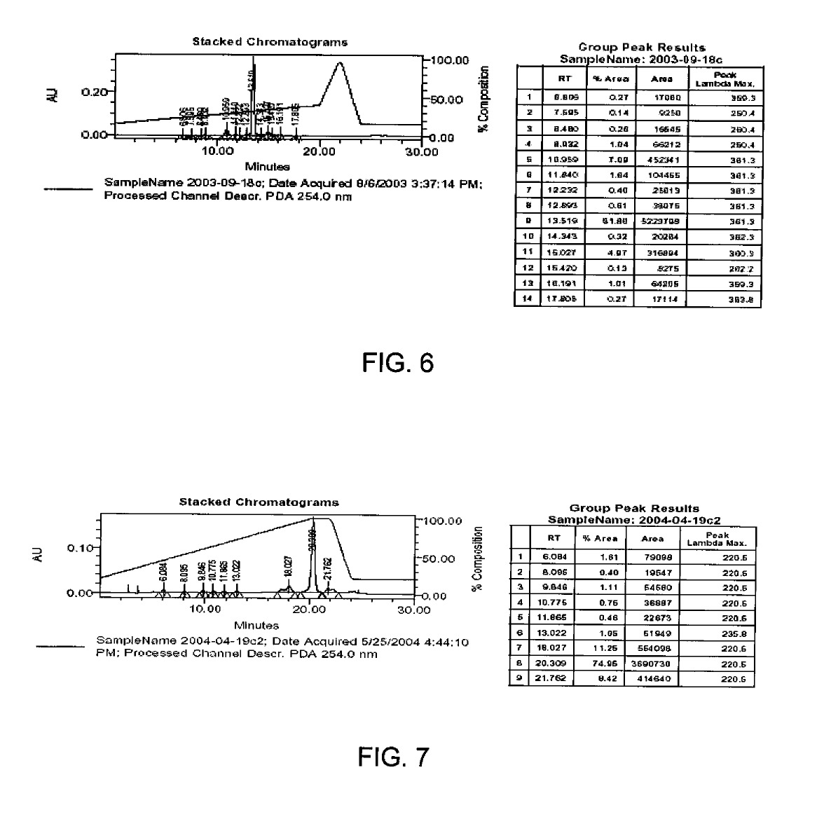

(m.) B₁₂-5′-OCONH(CH₂)₅COOSu (10) was synthesized as follows: To a stirring solution of B12-5′-OCONH(CH₂)₅COOH (0.6105 g, 0.4036 mmol, 1.0 eq) in DMSO (8 mL) was added SDPP (0.1549 g, 0.4461 mmol, 1.11 eq) and TEA (0.115 mL, 0.8183 mmol, 2.03 eq). The red solution was stirred at room temperature overnight. The reaction mixture was added to a stirring mixture of methylene chloride/ether (1:1, 100 mL). The resulting precipitate was collected, washed with acetone (4 mL×3), methylene chloride/ether (1:1, 4 mL×3), dried in vacuo. 0.6782 g of red powder was obtained. ES(+)−MS: 1610 (M+H). HPLC indicated the product to be about 82% pure (gradient: 15-40% B over 20 min) as shown in FIG. 6.

(n.) B₁₂-5′-OCONH(CH₂)₅CO-Phe-Lys(MMT)-PABC-7-Taxol-2′-MMT (11) was synthesized as follows: To a stirring solution of compound (10) (1.4251 g, ˜86% pure, 0.7614 mmol, 1.16 eq) in DMSO (20 mL) was added compound (7) (1.2015 g, 0.6590 mmol, 1.0 eq). The solution was stirred at room temperature for 1.5 hrs. Ether (90 ml) was added and an oily layer occurred. The ether was decanted and the residue was solidified with methylene chloride/ether (1:1, 80 mL). The resulting solid was collected, washed with ether (20 ml×3), air-dried and then washed with water (10 ml×3), dried in vacuo overnight. 1.2735 g (58.2%) of red powder were obtained. ESI(+)−MS: 1670 [(M+H+Na)²⁺]. HPLC indicated the product to be about 75% pure (gradient: 20 to 100% B over 20 min) as shown in FIG. 7.

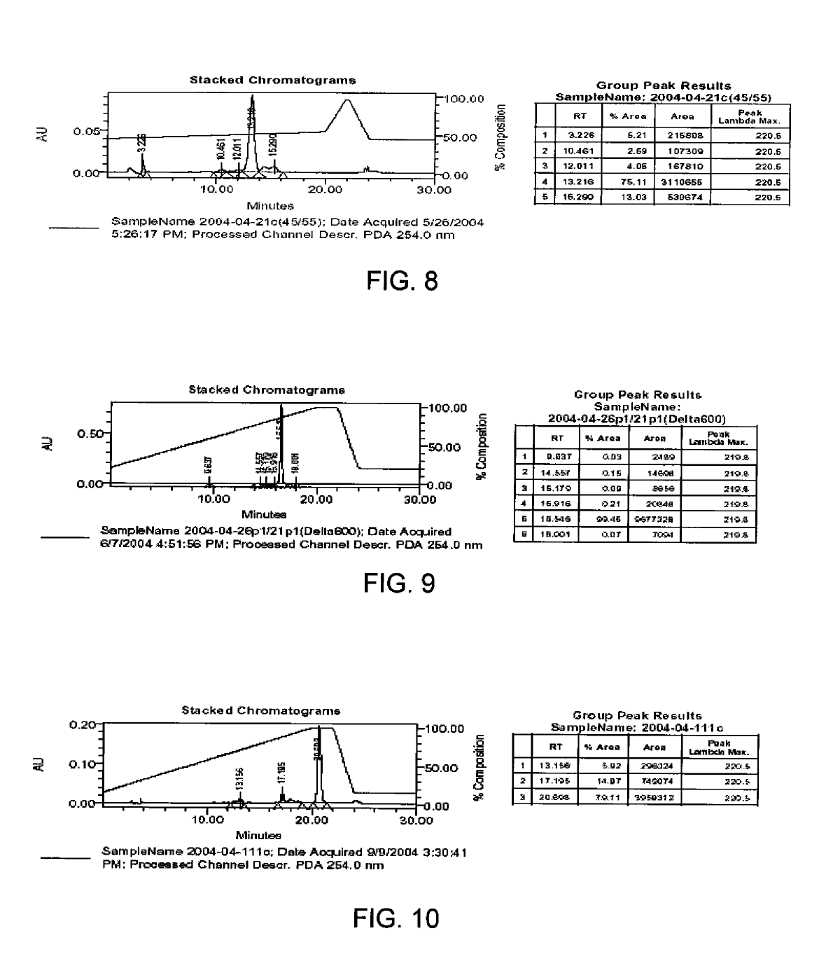

(o.) B₁₂-5′-OCONH(CH₂)₅CO-Phe-Lys-PABC-7-Taxol (12) was synthesized as follows: To a stirring suspension of compound (11) (1.2735 g, 0.3839 mmol, 1.0 eq) in methanol (40 ml), methylene chloride (40 ml) and water (40 ml), was added anisole (0.2 ml, 1.8310 mmol, 4.77 eq) and dichloroacetic acid (4.7 ml, 57.2187 mmol, 149.06 eq, ˜0.5 M final concentration). The solid dissolved and the mixture was stirred at RT for 1.5 hrs. The organic solvents were removed with rotary evaporator. The aqueous solution was decanted (HPLC didn't show much product in it). The sticky residue was dried by rinsing with ether (10 mL×4). The resulting solid was dissolved in methanol (10 ml), and added to stirring ether (90 ml). The resulting precipitate was collected, washed with ether (10 ml×3), dried in vacuo. 1.0900 g of red powder were obtained. HPLC indicated the product to be about 75% pure (gradient: 45 to 55% B over 20 min) as shown in FIG. 8.

(p.) The crude product was purified by HPLC as follows:
Column: Waters Delta-Pak C18 15 um (P/N: WAT038506) 25×300 mm.
Flow rate: 41 mL/min.
Solvents: 50 mM H₃PO₄/NH₄OH, pH3.0 (A) and 9:1 acetonitrile/water (B).
Gradient: 0-20 min, 40-50% B.
Sample was dissolved in 40% B in buffer A (10 mL), filtered through 0.45 um Nylon syringe filter. Seven injections were made (some impure fractions were re-purified).

The desired fractions were combined and desalted by Waters Sep-Pak tC18 cartridge (P/N: WAT043365). The product was lyophilized, giving 0.66 g (60%) of red powder. ESI(+)−MS: 2773.3 [(M+H)⁺, 9%], 1387.3 [(M+2H)²⁺, 100%], 1398.3 [(M+H+Na)²⁺, 7%]. HPLC indicated the product to be about 99.5% pure (gradient: 20 to 100% B over 20 min) as shown in FIG. 9.

The desalting protocol was as follows:
1. Wash the cartridge with methanol (3 cartridge volumes).
2. Wash the cartridge with water (3 cartridge volumes).
3. Load sample (dilute the collected HPLC fractions with 1 volume of water).
4. Wash with water (3 cartridge volumes).
5. Elute the product off the cartridge with methanol/water (9:1).
6. Remove methanol by rotary evaporator.
7. The residue was dissolved in water, filtered through 0.45 μm Nylon membrane filter and lyophilized.

### Example 2

### Preparation of Cobalamin-Taxol Bioconjugate

The synthesis process depicted in FIG. 3 was carried out as follows:

(a.) Paclitaxel was purchased from 210EC PX Pharm Ltd (UK). AAs and EEDQ were from Novabiochem. Fmoc-Phe-OSu was obtained from Advanced ChemTech. All other chemicals and solvents were from Acros, Aldrich, Sigma, Fluka, Fisher or VWR and used without further purification unless stated otherwise. Silica Gel 60 F₂₅₄ aluminium-backed TLC plates were obtained from VWR (P/N EM-5554-7). A Waters Alliance 2695 system including a 2996 PDA detector was used for analytical HPLC work. A Waters Delta 600 system including a 2996 PDA detector was used for preparative HPLC work. 50 mM H₃PO₄/NH₄OH, pH 3.0 (A) and 9:1 acetonitrile/water (B) were used as aqueous and organic eluants, respectively, unless stated otherwise. A Waters Delta-Pak C₁₈ 15 μm 100 Å 3.9×300 mm column (P/N WAT011797) with a 2 cm guard column (P/N WAT046880) and 1 mL/min flow rate were used for analytical work; a Waters Delta-Pak Radial Compression C₁₈ 15 μm 100 Å 25×300 mm column (P/N WAT011797) and 41 mL/min flow rate were used for preparative work. Mass spectra were acquired on an Applied Biosystems API 2000 electrospray mass spectrometer in positive ion mode.

(b.) Fmoc-Phe-OSu was synthesized as follows: To a suspension of Fmoc-Phe (7.7482 g, 0.0200 mol, 1.0 eq) and N-hydroxysuccinimide (2.4182 g, 0.0210 mol, 1.05 eq) in methylene chloride (150 ml) cooled in an ice bath, was added DCC (4.3440 g, 0.0211 mol, 1.05 eq). The mixture was stirred at room temperature overnight. The resulting DCU was removed by filtration and the filtrate was condensed and dried in vacuo to give 10.0798 g of white foam. R_{f}: 0.75 (5% CH₃OH/CH₂Cl₂).

(c.) Fmoc-Lys(MMT) (1) was synthesized as follows: To a stirred suspension of Fmoc-Lys (Novabiochem, 5.1067 g, 13.8618 mmol, 1.0 eq) in methylene chloride (75 ml) at room temperature was added trimethylsilyl chloride (Acros, 3.8 ml, 29.7312 mmol, 2.14 eq). The mixture was refluxed at 50° C. for 1 hr and the appearance of the solid in the reaction mixture changed. Then cooled in an ice bath, DIEA (7.5 ml, 43.0561 mmol, 3.11 eq) was added, the mixture became homogeneous, and followed by p-anisyldiphenylmethyl chloride (Acros, 4.4955 g, 14.5580 mmol, 1.05 eq). The orange-red solution was stirred at RT overnight (20 hrs). After removal of solvent, the residue was partitioned between ethyl acetate (200 ml) and pH5 buffer (0.05M phthalic acid, adjusted with 10N KOH to pH 5.0). The organic phase was washed with more pH5 buffer (50 ml×2), water (50 ml×1), brine (50 ml×2), dried over magnesium sulfate. Removal of solvent and being dried in vacuo gave a pale yellow foam (9.7336 g). TLC showed trace of impurities (R_{f}=0.45 for product, by 10% CH₃OH/CHCl₃). ¹H-NMR (CDCl₃, 300 MHz): OK (no TMS group).

(d.) Lys(MMT) (2) was synthesized as follows: To a stirred solution of Fmoc-Lys(MMT) (9.7336 g, assuming 13.8618 mmol) in 1:1 CH₂Cl₂/ACN (100 ml) at room temperature was added diethylamine (Acros, 100 ml). The mixture was stirred at RT for 1.5 hrs. After removal of solvent, the residue was flushed with acetonitrile at 60° C. (90 ml×2, being stirred 5 min), washed with acetonitrile (20 ml×3) and ether (20 ml×3). The solid was then dissolved as far as possible in 1:1 CH₂Cl₂/CH₃OH (200 ml) and some solid byproduct was removed by filtering through filter paper. After removal of solvent and being dried in vacuo, a pale yellow foam (4.7707 g, 82.2% based on Fmoc-Lys) was obtained. TLC(R_{f}=0, by 10% CH₃OH/CHCl₃) showed no starting material. (The solid recovered from the acetonitrile filtrate is the diethylamine salt). ES(+)−MS: 147 (Lys+1), 273 (MMT). ¹H-NMR (DMSO-d6, 300 MHz): OK.

(e.) Fmoc-Phe-Lys(MMT) (3) was synthesized as follows: To a stirred suspension of Fmoc-Phe-OSu (2.0702 g, 4.2728 mmol, 1.0 eq) and Lys(MMT) (1.7995 g, 4.2995 mmol, 1.01 eq) in DMF (30 ml) was added DIEA (1.5 ml, 8.6112 mmol, 2.02 eq). The solid dissolved gradually and the solution was stirred at RT overnight. The reaction mixture was partitioned between ethyl acetate (100 ml) and pH5 buffer (0.05M phthalic acid, adjusted with 10N KOH to pH 5.0, 200 ml). The aqueous solution was extracted with more ethyl acetate (50 ml×2). The combined organic phase was washed with brine (50 ml×3), dried over MgSO₄. After removal of solvent and being dried in vacuo, 3.3014 g (98.1%) of pale-yellow foam was obtained. TLC(R_{f}=0.43, by 10% CH₃OH/CHCl₃) showed a small impurity spot. ES(+)−MS: 788 (M+1), 810 (M+Na), 538 (M−MMT+Na), 273 (MMT). ¹H-NMR (DMSO-d6, 300 MHz): OK.

(f.) Fmoc-Phe-Lys(MMT)-PABOH (4) was synthesized as follows: To a stirred solution of Fmoc-Phe-Lys(MMT) (3.3014 g, 4.1898 mmol, 1.0 eq) and 4-aminobenzyl alcohol (Fluka, 0.6219 g, 5.0495 mmol, 1.21 eq) in CH₂Cl₂ (20 ml) was added 2-ethoxy-1-ethoxycarbonyl-1,2-dihydroquinoline (EEDQ, Novabiochem, 1.5589 g, 6.3037 mmol, 1.50 eq). The mixture was stirred at RT overnight. After removal of solvent, the residue was triturated with ether (50 ml). The mixture was left to stand at RT for 2 hours and then the solid was collected, washed with ether (15 ml×3), dried in vacuo. 2.1071 g (56.3%) of white solid was obtained. The ether filtrate was condensed. The residue was suspended in benzene (10 ml) and precipitated with hexane (10 ml). This process was repeated two more times. The resulting solid was collected, washed with benzene/hexane (1:1, 10 ml×3), dried in vacuo. Another 0.8864 g (23.7%) of white solid was obtained. Total yield: 80.0%. TLC(R_{f}=0.57, by 10% CH₃OH/CHCl₃) showed trace of impurity. ES(+)−MS: 893 (M), 915 (M+Na), 810 (M−PABOH+Na), 273 (MMT). ¹H-NMR (DMSO-d6, 300 MHz): OK.

(g.) Phe-Lys(MMT)-PABOH (5) was synthesized as follows: The solution containing Fmoc-Phe-Lys(MMT)-PABOH (1 g, 1.12 mmol) and DEA (8 mL) in 32 mL of CH₂Cl₂ was stirred for 3 h at rt. The mixture was concentrated to remove all solvent. The residue was dissolved in 2 mL of CH₂Cl₂ and 50 mL of CH₂Cl₂/hexane (1:9) was added to precipitate. The supernatant was poured out after stirring for a while. This precipitation was repeated three more times and residue was dried in vacuo to afford 703 mg (94%) of pale yellow powder. R_{f}: 0.2 (5% CH₃OH/CH₂Cl₂) ESI(+)−MS: 671.6 (M+1)⁺

(h.) Fmoc-AHA-OSu (6) was synthesized as follows: To a stirred solution of 6-aminohexanoic acid (1.7681 g, 5.0031 mmol, 1.0 eq) and N-hydroxysuccinimide (0.6068 g, 5.2724 mmol, 1.05 eq) in methylene chloride (75 ml) cooled in an ice-bath, was added DCC (1.0996 g, 5.3293 mmol, 1.07 eq). The mixture was stirred at RT overnight. The white solid was filtered off and washed with methylene chloride (10 ml×3). The is filtrate was condensed and the residue was re-dissolved in methylene chloride (10 ml). The white solid was removed by filtration and washed with methylene chloride (3 ml×3). The filtrate was evaporated and dried in vacuo, giving 2.436 g (108%) of white hygroscopic foam. It could be re-crystallized from isopropanol. R_{f}: 0.55 (5% CH₃OH/CH₂Cl₂).

An alternate synthesis process for this step is as follows: To a solution containing Fmoc-ε-Ahx-OH (4.82 g, 13.63 mmol), N-hydroxy succinimide (1.65 g, 14.34 mmol) in CH₂Cl₂ (200 mL) was added DCC (3.0 g, 14.45 mmol) at room temperature and stored overnight. Filtration to remove precipitate (DCU) and the residue was washed with CH₂Cl₂ (10 mL×2). The filtrate was concentrated and recrystallized in 35 mL of iso-propanol to afford 6.02 g (94.5%) of white powder.

(i.) Fmoc-AHA-Phe-Lys(MMT)-PABOH (7) was synthesized as follows: To a solution of Phe-Lys(MMT)-PABOH (500 mg, 0.745 mmol, 1.0 eq) and DIEA (0.143 mL, 0.82 mmol, 1.1 eq) in CH₂Cl₂ (8 mL) was added Fmoc-NH—(CH₂)₅COOSu (389 mg, 0.835 mmol, 1.12 eq). White precipitate formed after 1 hr. The precipitate was collected after overnight by filtration and washed with CH₂CH₂ (4 mL×3). The precipitate was dried in vacuo to afford 468 mg (62.4%) of white powder. R_{f}: 0.31 (5% CH₃OH/CH₂Cl₂).

(j.) PTX-2′-MMT (8) was synthesized as follows: To a stirred solution of paclitaxel (1.0033 g, 1.1749 mmol, 1.0 eq) and p-anisylchlorodiphenylmethane (2.8972 g, 9.3821 mmol, 7.98 eq) in CH₂Cl₂ (20 ml) was added pyridine (0.78 ml, 9.5651 mmol, 8.14 eq). The solution was stirred at RT overnight. After removal of solvent, the residue was dissolved in ethyl acetate (200 ml) and cold pH5 buffer (0.05M phthalic acid, adjusted with 10N KOH to pH 5.0, 100 ml). The organic phase was separated and washed with cold pH 5 buffer (100 ml×2), water (100 ml×1) and brine (100 ml×1), dried over MgSO₄. After removal of solvent, the residue was purified by silica column (5×10 cm, packed with 4:1 hexane/ethyl acetate; Sample was dissolved in ethyl acetate, adsorbed to 10 g of silica gel, air-dried and loaded onto the column), eluting with hexane/ethyl acetate (1:1, 160 ml; 2:3, 400 ml), giving 1.2451 g (94.1%) of white solid. R_{f}: 0.52 (2:3 hexane/ethyl acetate).

(k.) Fmoc-AHA-Phe-Lys(MMT)-PABC-7-PTX-2′-MMT (9) was synthesized as follows: To an ice-cooled solution of PTX-2′-MMT (0.4539 g, 0.4030 mmol, 1.0 eq) in methylene chloride (4 mL) was added DIEA (0.07 ml, 0.4019 mmol, 1.00 eq), pyridine (0.033 ml, 0.4047 mmol, 1.00 eq) and then diphosgene (0.025 ml, 0.2072 mmol, 0.51 eq). The ice bath was removed and the solution was stirred at RT for 1.5 hours. The resulted solution was added to a suspension of Fmoc-AHA-Phe-Lys(MMT)-PABOH (0.4039 g, 0.4014 mmol, 1.00 eq) and DIEA (0.07 ml, 0.4019 mmol, 1.00 eq) in methylene chloride (4 ml). The suspension was stirred at RT overnight. The reaction mixture was filtered, washed with ethyl acetate (5 ml×3) and water (20 ml×3), dried in vacuo, giving 0.2027 g of compound (7). The filtrate was diluted with ethyl acetate (100 ml), washed with pH 5 buffer (0.05M phthalic acid, adjusted with 10N KOH to pH 5.0, 50 ml×3), water (50 ml×1) and brine (50 ml×1), dried over MgSO₄. After removal of solvent, the residue was purified by silica column (2.4×20 cm, packed with 2:1 methylene chloride/ethyl acetate, sample dissolved in 2:1 methylene chloride/ethyl acetate), eluting with methylene chloride/ethyl acetate (3:2), giving 0.1994 g (23%) of white solid. R_{f}: 0.17 (3:2 methylene chloride/ethyl acetate). [0.1752 g of compound (8) was recovered].

(l.) AHA-Phe-Lys(MMT)-PABC-7-PTX-2′-MMT (10) was synthesized as follows: To a stirred solution of Fmoc-AHA-Phe-Lys(MMT)-PABC-7-PTX-2′-MMT (110.3 mg, 51.4 umol, 1.0 eq) in THF (2 ml) was added 1,8-diazabicyclo[5.4.0]undec-7-ene (DBU, 0.02 mL, 132.7 umol, 2.58 eq, final concentration: 1%). The solution was stirred at RT for 10 minutes. The reaction mixture was added to stirred hexane (40 mL). The resulting precipitate was collected, washed with hexane (5 mL×3), dried in vacuo, giving 91.0 mg (91.4% yield) of white solid.

(m.) B₁₂-5′-OCO(1,2,4-triazole) (11) was synthesized as follows: To a stirred solution of cyanocobalamin (2.0380 g, 1.5036 mmol, 1.0 eq) in DMSO (30 ml) was added 1,1′-carbonyldi(1,2,4-triazole) (0.3759 g, 2.2903 mmol, 1.52 eq). The mixture was stirred at RT for 10 min. HPLC indicated about 12% of starting cyanocobalamin left (Tr=12.77 min for SM, Tr=13.99 min for product (8), 10 to 30% B over 20 min; A: 0.1% HOAc/water; B: acetonitrile). At time point of 30 min, the reaction mixture was added to stirred mixture of CH₂Cl₂/ether (1:1, 200 ml). The resulting precipitate was collected, washed with acetone (50 ml×3) and ether (50 ml×1), dried in vacuo, giving 2.3706 g of red powder. HPLC showed the product to be about 81.5% pure as shown in FIG. 10.

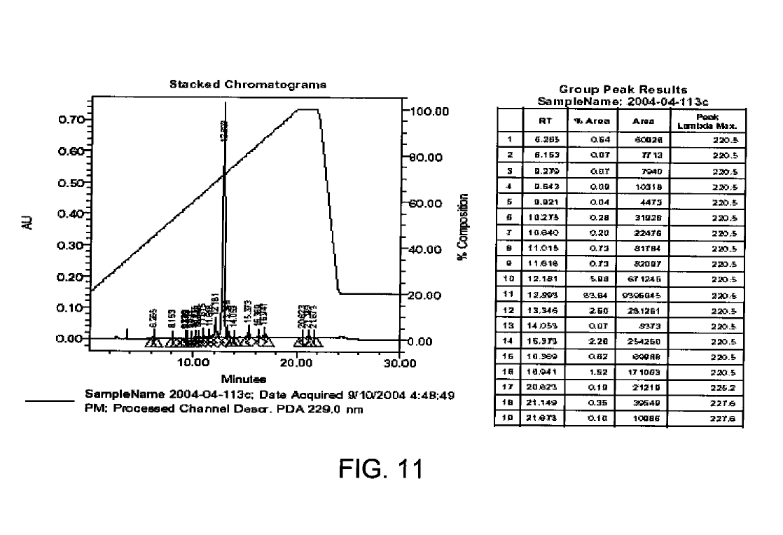

(n.) B₁₂-5′-AHA-Phe-Lys(MMT)-PABC-7-PTX-2′-MMT (12) was synthesized as follows: To a stirred solution of compound (11) (0.1513 g, ˜86% pure, 0.0897 mmol, 1.62 eq) in DMSO (1 mL) was added compound (10) (0.107 g, 0.0553 mmol, 1.0 eq). The solution was stirred at room temperature for 30 min. Then the reaction mixture was added to stirred ether (40 mL). The ether was decanted and the sticky residue was triturated with methylene chloride/ether (1:1, 15 mL). The resulting solid was collected, washed with methylene chloride/ether (1:1, 5 ml×3), and water (5 ml×3), dried in vacuo, giving 0.1544 g (84.2% yield) of red powder. HPLC indicated it to be about 79% pure (Tr=20.698 min, 20 to 100% B over 20 min) as shown in FIG. 11.

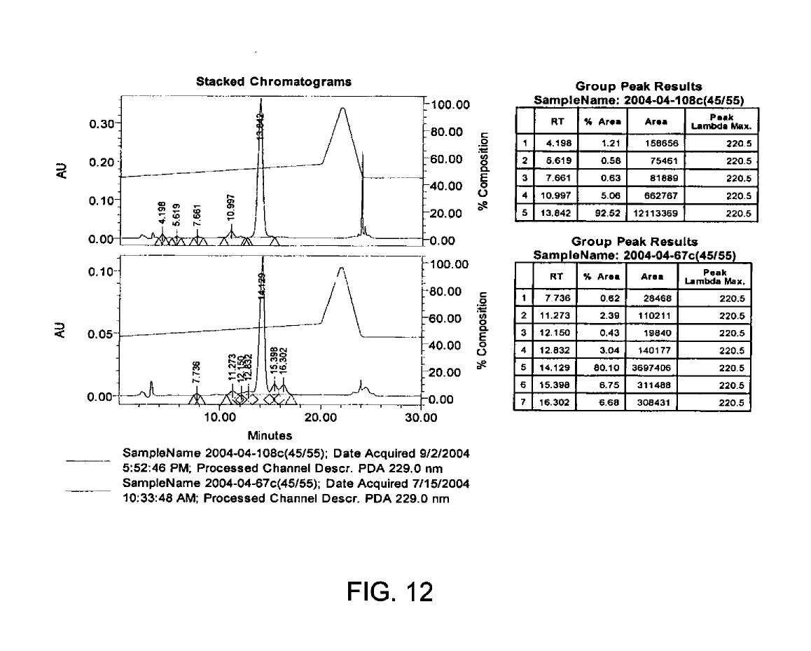

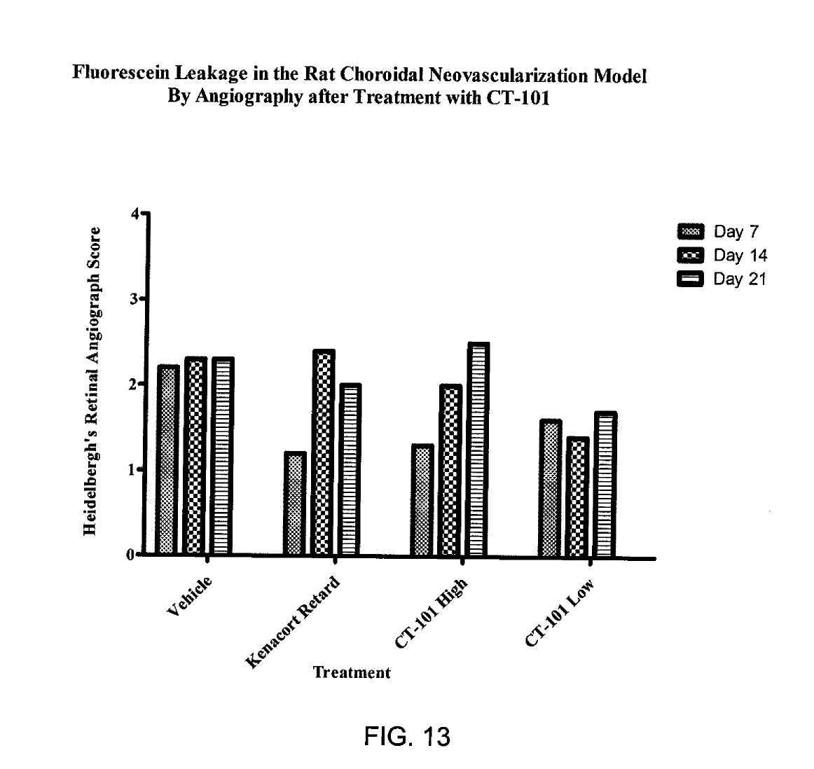

(o.) B₁₂-5′-AHA-Phe-Lys-PABC-PTX-7 (13) was synthesized as follows: To a stirred suspension of compound (12) (0.1544 g, 46.5 umol, 1.0 eq) in methanol (5 ml), methylene chloride (5 ml) and water (5 ml), was added anisole (0.1 ml) and dichloroacetic acid (0.6 ml, about 0.5 M final concentration). The solid dissolved and the mixture was stirred at RT for 2 hrs. The reaction mixture was diluted with water (10 ml) and the organic solvents were removed with a rotary evaporator. The aqueous solution was decanted (HPLC didn't show much product in it). The sticky residue was dried via rinsing with ether (2 mL×4). The resulted solid was dissolved in methanol (2 ml), and added to stirred ether (40 ml). The resulting precipitate was collected, washed with methylene chloride (2 ml×3) and ether (2 ml×2), dried in vacuo. 122 mg (90% yield) of red powder were obtained. HPLC indicated the product to be about 83.6% pure (Tr=12.89 min, 20 to 100% B over 20 min) as shown in FIG. 12.

### Example 3

### Choroidal Neovascularization Model

Groups of rats were neovascularized by argon laser scarring on an eye. At the same time (day 0), the eye was immediately treated with the cobalamin-paclitaxel bioconjugate (CT-101) prepared in accordance with Example 1. Two concentrations, one higher and one lower, were tested. The treatment regimen also included a vehicle and KENACORT® RETARD (4% triamcinolone acetonide), as a positive control. The treated eyes were evaluated for inhibition of neovascularization on days 7, 14 and 21 by infusing the eye with fluorescein and scoring the leakage from each spot. The leakage of fluorescein on the photographies of the angiograms from each spot were evaluated by two examiners using an HRA (Heidelberg's Retinal Angiograph) in a masked fashion and graded as follows:

Score 0: no leakage;

Score 1: slightly stained;

Score 2: moderate stained;

Score 3: strongly stained.

As shown in FIG. 1, the bioconjugate provided a marked angiogenic effect relative to the vehicle over 7 days, and was comparable in effect to the positive control. Unlike the positive control, both bioconjugate treatments showed a measurable effect as late as Day 14.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method of treating an eye disease, comprising administering a bioconjugate to a subject to treat the eye disease, wherein the bioconjugate comprises a taxane selected from the group consisting of paclitaxel or docetaxel covalently bonded to the cobalamin vitamin B12.

2. The method of claim 1, wherein the bioconjugate comprises a linker covalently bonded to a 5′-OH moiety of the cobalamin and the taxane is covalently bonded to the linker; the taxane is cleavable from the linker and/or the linker is cleavable from the drug by an intracellular enzyme; and the conjugate is adapted for transport across a cellular membrane after complexation with transcobalamin.

3. The method of claim 2, wherein the linker is cleavable by way of one of a class of intracellular enzymes, said class of enzymes selected from the group of cathepsin, endo enzyme, glycosidase, metalloprotease, ribozyme, protease, esterase, and amidase.

4. The method of claim 1, wherein the conjugate possesses one or more protecting groups.

5. The method of claim 1, wherein the bioconjugate is dissolved in an aqueous solution prior to administration.

6. The method of claim 1, wherein the bioconjugate has a water solubility of at least 50 mg/ml.

7. The method of claim 1, wherein the bioconjugate has a water solubility of at least 100 mg/ml.

8. The method of claim 1, wherein the step of administering achieves serum levels of about 0.1 ng/ml to about 20,000 ng/ml of the taxane in the subject.

9. The method of claim 1, wherein the step of administering is by ocular administrations.

10. The method of claim 1, wherein the taxane portion of the bioconjugate is administered at about 1 mg/kg/day to about 10 mg/kg/day.

11. The method of claim 1, wherein the taxane portion of the bioconjugate is administered at about 2 mg/kg/day to about 6 mg/kg/day.

12. The method of claim 1, wherein the eye disease is selected from the group consisting of age-related macular degeneration, proliferative diabetic retinopathy, nonproliferative diabetic retinopathy, retinopathy of prematurity, corneal graft rejection, neovascular glaucoma, rubeosis, pterygia, abnormal blood vessel growth of the eye, uveitis, dry-eye syndrome, post-surgical inflammation and infection of the anterior and posterior segments, angle-closure glaucoma, open-angle glaucoma, post-surgical glaucoma procedures, exopthalmos, scleritis, episcleritis, Grave's disease, pseudotumor of the orbit, tumors of the orbit, orbital cellulitis, blepharitis, intraocular tumors, retinal fibrosis, vitreous substitute and vitreous replacement, iris neovascularization from cataract surgery, macular edema in central retinal vein occlusion, cellular transplantation (as in retinal pigment cell transplantation), cystiod macular edema, psaudophakic cystoid macular edema, diabetic macular edema, pre-phthisical ocular hypotomy, proliferative vitreoretinopathy, extensive exudative retinal detachment (Coat's disease), diabetic retinal edema, diffuse diabetic macular edema, ischemic opthalmopathy, pars plana vitrectomy (for proliferative diabetic retinopathy), pars plana vitrectomy for proliferative vitreoretinopathy, sympathetic ophthalmia, intermediate uveitis, chronic uveitis, retrolental fibroplasia, fibroproliferative eye diseases, acquired and hereditary ocular conditions such as Tay-Sach's disease, Niemann-Pick's disease, cystinosis, corneal dystrophies, and combinations thereof.

13-52. (canceled)
